(19) United States
(12) Patent Application Publication
(10) Pub. No.: US 2012/0040333 A1
(43) Pub. Date: Feb. 16, 2012

(54) Methods to Distinguish Different Disaccharide Products after Digestion with Heparinases

(75) Inventor: David Landry, Essex, MA (US)

(73) Assignee: NEW ENGLAND BIOLABS, INC., Ipswich, MA (US)

(21) Appl. No.: 13/166,254

(22) Filed: Jun. 22, 2011

Related U.S. Application Data
(60) Provisional application No. 61/373,032, filed on Aug. 12, 2010.

(51) Int. Cl. C12Q 1/527

(52) U.S. Cl. 435/4

(57) ABSTRACT

Methods are provided for determining stereochemistry for a component of a composition where the composition includes a glycosaminoglycan (GAG) where the method includes (a) cleaving the GAG with a lyase; (b) separating the products of the cleaved GAG by chromatography in the presence of one or more solvents wherein at least one solvent comprises an organic acid or organic acid derivative in an amount of no more than 50% v/v of solvent; and determining the stereochemistry of the component.

### CROSS REFERENCE

This application claims priority from U.S. provisional application No. 61/373,032 filed Aug. 12, 2010, herein incorporated by reference.

### BACKGROUND OF THE INVENTION

Glycosaminoglycans (GAGs) are saccharide polymers which interact in vivo with molecules of biological importance such as growth factors, coagulation factors, viral proteins, cell adhesion molecules, chemokines, and proteases (Yamada et al. Current Drug Discovery Technologies 5:289-301(2008)). GAGs contain regions of structural diversity called saccharide domains which are recognized by and interact with specific molecules. The size, charge density, saccharide residues, and sequence of saccharide domains are factors which affect the specificity of the GAGs' interactions. (Taylor and Gallo FASEB Journal 20:9-22 (2006); and Saisekharan et al. Annual Reviews of Biomedical Engineering 8:181-231 (2006)). Uronic acid is a common saccharide residue and is found in two forms: glucuronic acid (GlcA) and iduronic acid (IdoA). Glucuronic acid differs from iduronic acid in the spatial orientation of its C5 carboxyl group. The difficulty of distinguishing IdoA and GlcA within a GAG-derived oligosaccharide has been a limiting step in the development of high throughput methods to determine the fine structure of saccharide domains.

### SUMMARY OF THE INVENTION

An embodiment of the invention provides a method for determining a stereochemistry for a component of a composition comprising a GAG, wherein the method includes: (a) cleaving the GAG with a lyase; (b) separating the products of the cleaved GAG by chromatography in the presence of one or more solvents wherein at least one solvent comprises an organic acid or organic acid derivative in an amount of no more than 50% v:v of solvent; (c) determining the stereochemistry for a component of the composition comprising the GAG; and (d) optionally subjecting the composition to mass spectrometry (MS).

The GAG of the above-described embodiment of the invention may contain modifications such as sulfation, carboxylation or acetylation.

The component of the above-described embodiment of the invention may be a disaccharide containing uronic acid or hexosamine or both.

The composition of the above-described embodiment of the invention may be isolated from a tissue of an organism or synthetic.

The composition of the above-described embodiment of the invention may be a polysaccharide, glycoprotein or glycolipid.

Chromatography methods according to the above-described embodiment of the invention may include column chromatography, thin layer chromatography (TLC) and high-performance liquid chromatography (HPLC). The chromatography media is optionally silica, silica gel or controlled pore glass.

The at least one solvent containing an organic acid or organic acid derivative of the above-described embodiment of the invention may have an acid disassociation constant (pKa) of less than 2.5 and no more than 6.5. Examples of organic acids include acetic acid, formic acid and propanoic acid. The at least one solvent may also contain one or more alcohols; optionally, the at least one solvent is at least 50% alcohol by volume. Optionally one of the solvents may be an acetonitrile solvent.

### BRIEF DESCRIPTION OF THE DRAWINGS

FIG. 1 shows the cleavage products resulting from digestion of heparan sulfate with Heparinase II. The bonds that are cleaved are indicated by arrows. The disaccharide product labeled “a” is 4-O-(4-deoxy-2-O-sulphato-β-D-glucuro-hex-3-enopyranosyluronic acid)-(2-acetamido-2-deoxy-2-sulfamino-6-O-sulphato-D-glucopyranose abbreviated herein as β-D-GlcA2S-(1-4)-D-GlcNS6S. The disaccharide product labeled “b” is 4-O-(4-deoxy-2-O-sulphato-α-L-iduro-hex-3-enopyranosyluronic acid)-(2-acetamido-2-deoxy-2-sulfamino-6-β-sulphato-D-glucopyranose), abbreviated herein as α-L-IdoA2S-(1-4)-D-GlcNS6S.

FIGS. 2A-2B show the cleavage specificities of Heparinases I, II and III. The linkage cleaved is indicated by the thick arrow. The double-headed arrow indicates that the uronic acid may be either GlcA or IdoA. The brackets represent variable sulfation sites.

FIG. 2A shows the cleavage specificity of Heparinase I. Heparinase I cleaves the glycosidic linkage between N-sulfated glucosamine and 2-O-sulfated uronic acid.

FIG. 2B shows the cleavage specificity of Heparinase II. Heparinase II will cleave the glycosidic linkage between N-sulfated glucosamine and uronic acid; either the uronic acid or glucosamine may have O-sulfation.

FIG. 2C shows the substrate specificity of Heparinase III. Heparinase III cleaves the glycosidic linkage between N-sulfated or N-acetylated glucosamine and GlcA when the glucosamine is also 6-sulfated; when the substrate glucosamine lacks N-sulfation and 6-sulfation Heparinase III cuts IdoA and GlcA.

FIGS. 3A-3B show the chemical structures of the 7-amino-4-methylcoumarin (AMC)-labeled, highly sulfated GAG tetramer (FIG. 3A) and a TLC plate spotted with the products of titration of the Heparinases I, II & III on a highly sulfated GAG tetramer covalently labeled with (AMC) (FIG. 3B). In FIG. 3B, “i.” indicates the position of α-L-IdoA-(1-4)-D-GlcNS6S-AMC, “ii.” indicates the position of α-L-IdoA2S-(1-4)-D-GlcNS6S-AMC, and “iii.” indicates the position of β-D-GlcA2S-(1-4)-D-GlcNS6S-AMC.

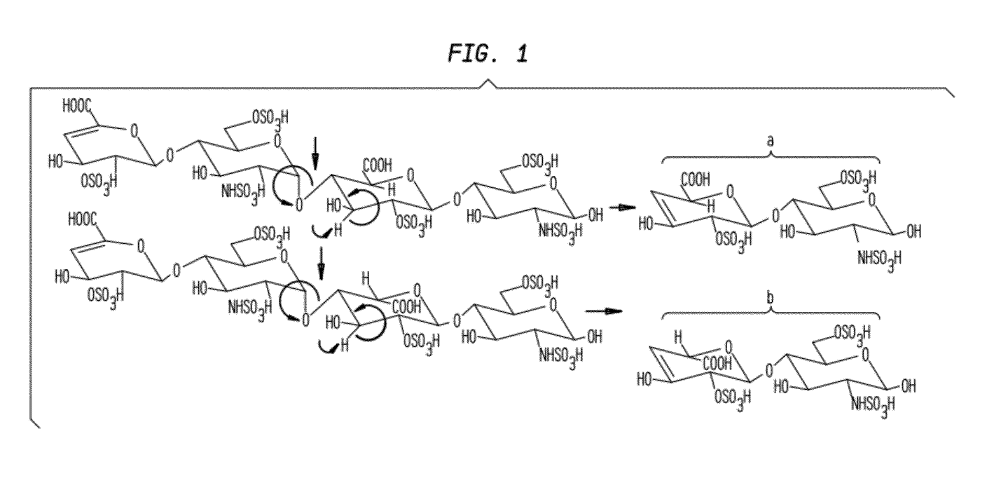

Lanes 1 and 12 contain the commercially prepared mass spectrometry standard, Disaccharide I-S (DI-S) (Sigma-Aldrich, Inc., St. Louis, Mo.), covalently bound to AMC.

Lanes 2 and 13 contain the commercially prepared mass spectrometry standard Disaccharide II-S (DII-S) (Sigma-Aldrich), covalently bound to AMC.

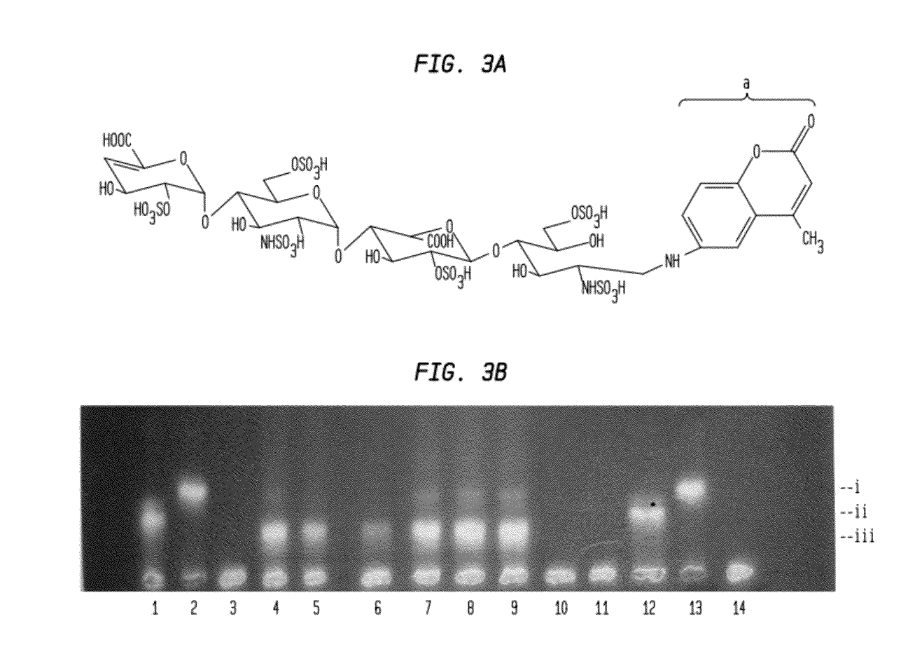

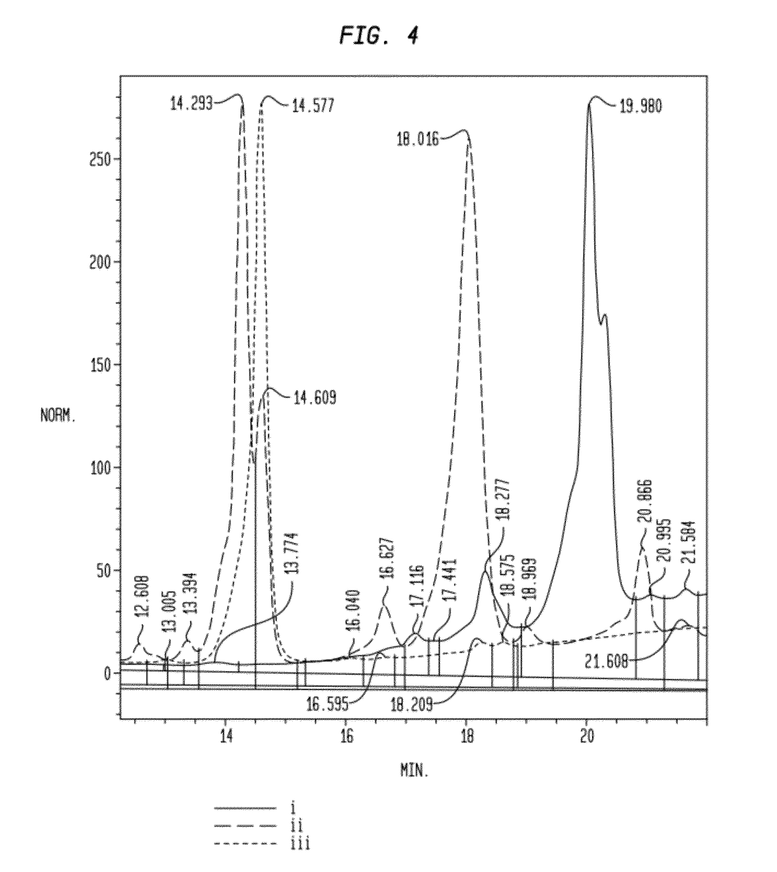

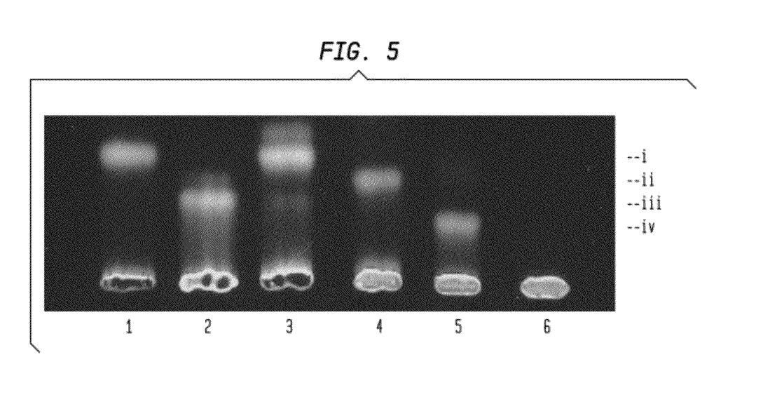

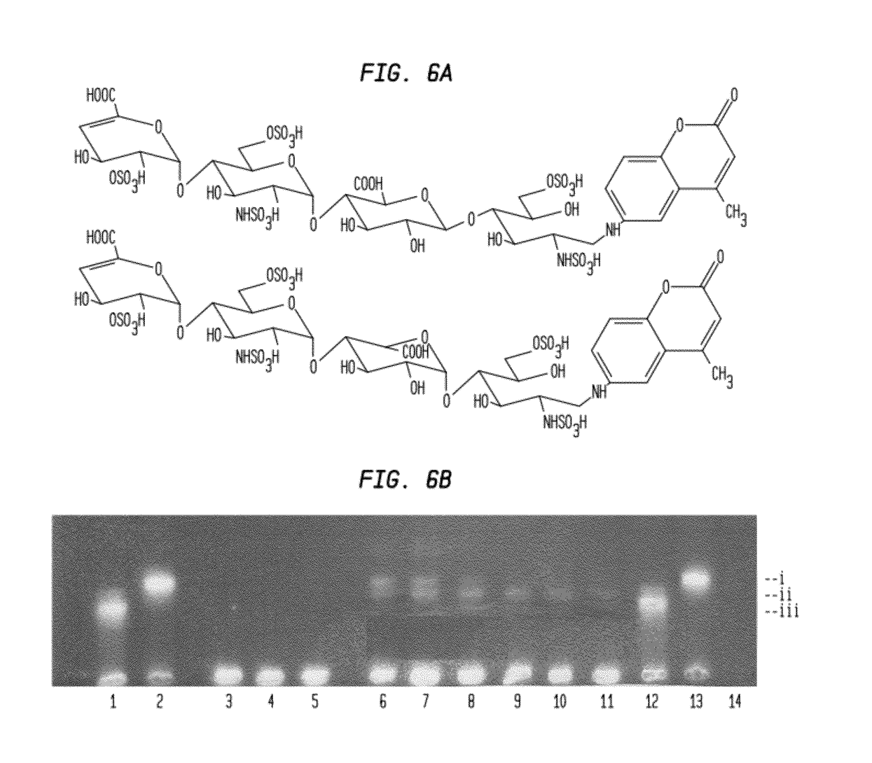

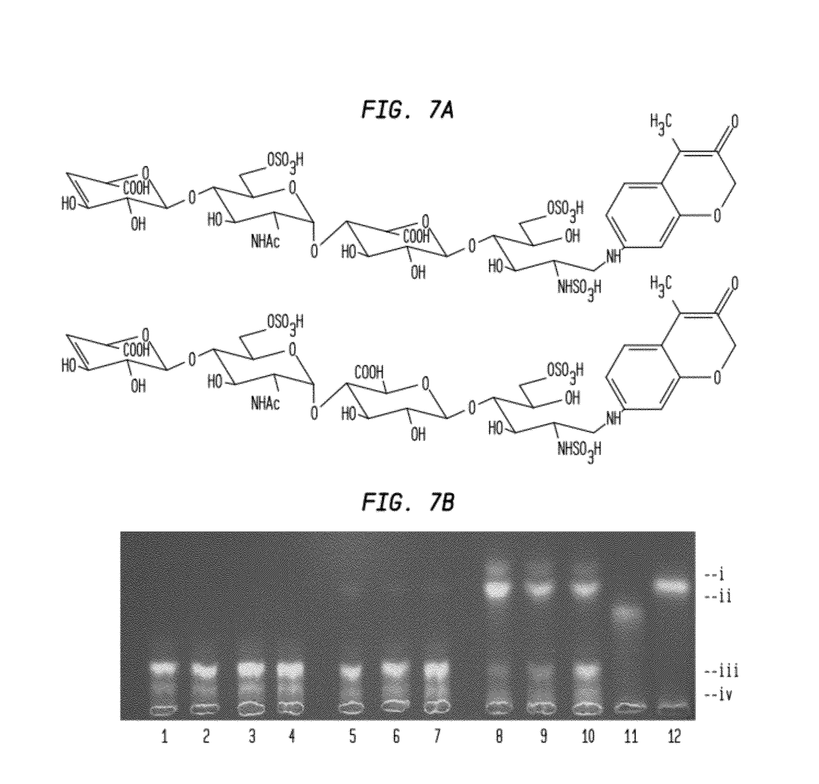

Lane 3 contains the heparin sulfate tetramer illustrated in FIG. 3A.

Lane 4 contains heparin sulfate tetramer treated with 1.0 μl Heparinase I (17.0 IU/ml, IBEX).

Lane 5 contains the heparin sulfate tetramer treated with 0.5 μl Heparinase I.

Lane 6 contains the heparin sulfate tetramer treated with 0.2 5 μl Heparinase I.

Lane 7 contains the heparin sulfate tetramer treated with 1.0 μl Heparinase II (15.2 IU/ml, IBEX Technologies, Inc., Montreal, Quebec).

Lane 8 contains the heparin sulfate tetramer treated with 0.5 μl Heparinase II.

Lane 9 contains the heparin sulfate tetramer treated with 0.25 μl Heparinase II.

Lane 10 contains the heparin sulfate tetramer treated with 1.0 μl Heparinase III (17.2 IU/ml, IBEX).

Lane 11 contains the heparin sulfate tetramer treated with 0.5 μl Heparinase III.

Lane 14 contains the heparin sulfate tetramer treated with 0.25 μl Heparinase III.

FIG. 4 shows the overlay of the chromatograms produced by the mass spectrometry of the hexa-sulfated GAG tetramer of FIG. 3A, the hexa-sulfated GAG tetramer after digestion with Heparinase II and the mass spectrometry standard DI-S. The AMC-labeled products of Heparinase digestion have masses nearly identical masses to the DI-S-AMC standard.

The solid line “i.” indicates the chromatogram of AMC-labeled hexa-sulfated GAG tetramer as shown in FIG. 3A. The peak at 19.980 indicates the AMC labeled hexa-sulfate tetramer whose structure is represented by α-L-IdoA2S-(1,4)-α-D-GlcNS6S-β-D-GlcA2S-(1-4)-D-GlcNS6S-AMC and which has a theoretical mass of 655.49 (654.2, unreduced) and an observed mass of 654.5 (Z+2).

The dashed line “ii.” indicates the chromatogram of the AMC-labeled hexa-sulfated GAG tetramer after digestion with Heparinase II. The peak at 14.293 indicates the cleavage product β-D-GlcA2S-(1-4)-D-GlcNS6S-AMC which has a theoretical mass of 367.04 and an observed mass of 366.00 (Z+2). The peak at 14.609 indicates the cleavage product α-L-IdoA2S-(1-4)-D-GlcNS6S-AMC which has a theoretical mass of 367.04 and an observed mass of 366.00 (Z+2). The peak at 18.016 indicates the product α-L-IdoA2S-(1,4)-D-GlcNS6S which as theoretical masses of 575.96 corresponding to an observed mass of 576.0 (Z+1) and 287.47 corresponding to 287.5 (Z+3).

The dotted line “iii.” indicates chromatogram of AMC-labeled DI-S. The peak at 14.577 indicates AMC-labeled DI-S whose structure is represented by α-L-IdoA2S-(1-4)-D-GlcNS6S-AMC and which has a theoretical mass of 367.04 and an observed mass of 367.00 (Z+2).

FIG. 5 shows the results of TLC of the labeled tetrasaccharide (FIG. 3A) after treatment with Heparinase II (IBEX), Heparinase II and 2-O-sulfatase (Sigma-Aldrich) and DII-S and DI-S (Sigma-Aldrich); DI-S which differs from DII-S only in that it contains 2-O-sulfation is shown after treatment with 2-O-sulfatase in Lane 3. “i.” shows the position of α-L-IdoA-(1-4)-D-GlcNS6S, “ii” shows the position of β-D-GlcA-(1-4)-D-GlcNS6S-AMC, “iii” shows the position of α-L-IdoA2S-(1-4)-D-GlcNS6S, “iv” shows the position of β-D-GlcA2S-(1-4)-D-GlcNS6S-AMC.

Lane 1 contains DII-S covalently bound to AMC.

Lane 2 contains DI-S covalently bound to AMC.

Lane 3 contains DI-S covalently bound to AMC after digestion with 2-O-sulfatase.

Lane 4 contains the product of digestion of the labeled tetrasaccharide (FIG. 3A) with Heparinase II and 2-O-sulfatase.

Lane 5 contains the product of digestion of the labeled tetrasaccharide with Heparinase II.

Lane 6 contains the labeled tetrasaccharide (FIG. 3A).

FIG. 6A shows the chemical structures of the AMC-labeled pentasulfated GAG tetramers which were digested with Heparinase I, II or III before being spotted on the TLC plate illustrated in FIG. 6B.

FIG. 6B shows the resolution on a TLC plate of the cleavage products resulting from the treatment of the AMC oligo-GAGs shown in FIG. 6A with Heparinases I, II or III (IBEX). “i.” shows the position of α-L-IdoA-(1-4)-D-GlcNS6S. “ii” shows the position of β-D-GlcA-(1-4)-D-GlcNS6S-AMC. “iii” shows the position of α-L-IdoA2S-(1-4)-D-GlcNS6S.

Lanes 1 and 12 contain AMC-labeled DI-S.

Lanes 2 and 13 contain AMC-labeled DII-S.

Lane 3 contains the tetramer mixture treated with 1.0 μl Heparinase I.

Lane 4 contains the tetramer mixture treated with 0.5 μl Heparinase I.

Lane 5 contains the tetramer mixture treated with 0.25 μl Heparinase I.

Lane 6 contains the tetramer mixture treated with 1.0 μl Heparinase II.

Lane 7 contains the tetramer mixture treated with 0.5 μl Heparinase II.

Lane 8 contains the tetramer mixture treated with 0.25 μl Heparinase II.

Lane 9 contains the tetramer mixture treated with 1.0 μl Heparinase III.

Lane 10 contains the tetramer mixture treated with 0.5 μl Heparinase III.

Lane 11 contains the tetramer mixture treated with 0.25 μl Heparinase III.

FIG. 7A shows the chemical structures of the AMC-labeled tetrasaccharides used in the TLC shown in FIG. 7B.

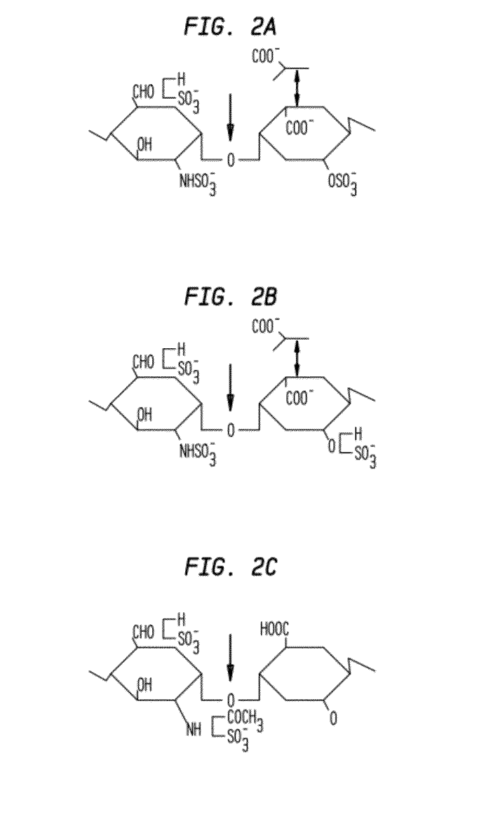

FIG. 7B shows the titration of the three Heparinases (I, II and III) on the moderately sulfated GAG tetramer mixture shown in FIG. 7A “i.” indicates the position of β-D-GlcA-(1-4)-D-GlcNS-AMC. “ii.” indicates the position of α-L-IdoA-(1-4)-D-GlcNS6S. “iii.” indicates the position of α-L-IdoA2S-(1-4)-α-D-GlcNAc-α-L-IdoA-(1-4)-D-GlcNS6S-AMC (FIGS. 7A, 2) and “iv.” indicates the position of α-L-IdoA2S-(1-4)-α-D-GlcNAc6S-β-D-GlcA-(1-4)-D-GlcNS-AMC (FIG. 7A, 1).

Lane 1 contains undigested tetramer mixture.

Lane 2 contains the tetramer mixture treated with 1.0 μl Heparinase I (17.0 IU/ml, IBEX).

Lane 3 contains the tetramer mixture treated with 0.5 μl Heparinase I.

Lane 4 contains the tetramer mixture treated with 0.25 μl Heparinase I.

Lane 5 contains the tetramer mixture treated with 1.0 μl Heparinase II (15.2 IU/ml, IBEX).

Lane 6 contains the tetramer mixture treated with 0.5 μl Heparinase II.

Lane 7 contains the tetramer mixture treated with 0.25 μl Heparinase II.

Lane 8 contains the tetramer mixture treated with 1.0 μl Heparinase III (17.2 IU/ml, IBEX).

Lane 9 contains the tetramer mixture treated with 0.5 μl Heparinase III.

Lane 10 contains the tetramer mixture treated with 0.25 μl Heparinase III.

Lane 11 contains AMC-labeled DI-S.

Lane 12 contains AMC-labeled DII-S.

### DETAILED DESCRIPTION OF THE EMBODIMENTS

Embodiments of the invention provide a means for identification of specific components within oligo-GAGs by treating a prepared sample with one or more lyases and separating the saccharide products resulting from lyase digestion (referred to herein as “cleavage products”) via chromatography in the presence of a solvent comprising an organic acid in no more than 50% v:v of the solvent.

In particular embodiments of the invention the sulfation or carboxylation state and stereochemistry of a component of an oligo-GAG may be identified based on the known specificity of a single lyase or by comparing the cleavage products of lyases with different, known specificities. In additional embodiments of the invention, the components of an oligo-GAG may be homogeneous or heterogeneous.

In an embodiment of the invention, the sulfation, carboxylation or acetylation state and stereochemistry of components of a composition comprising an oligo-GAG may be identified based on the known specificity of a single lyase; an example is shown in FIG. 5. Cleavage products that are separated by chromatography may be compared to oligo-GAG standards which differ by a known modification. Additionally, oligo-AG standards and cleavage products may be treated with one or more enzymes which act upon modifications at certain positions on the molecules, e.g., see Example 4. Modifying enzymes include O-sulfatases, N-sulfatases, epimerases and carboxy-lyases.

In an embodiment of the invention, the components of a composition comprising an oligo-GAG can be identified as either GlcA or IdoA by comparing the cleavage products of lyases with different, known specificities; examples include the reactions shown in FIGS. 3B, 6B and 7B.

A further embodiment of the invention is the identification of the sulfation, carboxylation or acetylation state and stereochemistry of the components of a composition comprising an oligo-GAG wherein the composition contains a heterogeneous mixture of oligosaccharides, e.g., see FIGS. 6B and 7B. The cleavage products resulting from treatment with lyases of differing specificities may be compared to identify the characteristics of the components.

“Component” as used herein refers to individual sugars and sugar-derivatives which make up the repeating units of a GAG. Examples of sugar-derivatives include hexosamines such as glucosamine and galactosamine and uronic acids such as GlcA and IdoA. Cleavage products comprising 2, 4, 6, 8, 10 or 12 contiguous components may be separated in embodiments of the present invention.

For example, in FIG. 6B the stereochemistry of cleavage products containing two components (a diamer) is determined, while in FIG. 7B the stereochemistry of oligo-GAGs comprising four components (a tetramer) is resolved.

“Stereochemistry” as used herein refers to the relative spatial positioning of functional groups within a molecule of interest, for example the cis or trans orientation of the C5 carboxyl group of uronic acid in relation to the anomeric carbon. The cis or trans orientations are alternately referred to herein as the axial and equatorial positions, respectively. The anomeric carbon is the additional asymmetric center at the carbonyl carbon atom which results from the cyclization of monosaccharides.

Oligo-GAGs may be synthetic or isolated from a variety of natural sources. Natural sources of GAGs include mammalian tissues and more particularly human tissues including cornea, cartilage, bone, skin, mucous membrane, lung, blood and blood vessels, muscle cells, extracellular matrixes and mast cells. A variety of chemical and enzymatic methods for artificial synthesis of GAG oligos have been developed. For a review of methods see Karst & Roberts (Current Medicinal Chemistry 10(19):1993-2031(2003)).

Examples of GAGs include keratan sulfate (consisting of the repeating unit comprising galactose linked to glucosamine), hyaluronic acid (repeating unit comprising GlcA linked to glucosamine), chondroitin sulfate/dermatan sulfate (repeating unit comprising GlcA or IdoA linked to galactosamine) and heparin/heparan sulfate (repeating unit comprising IdoA or GlcA linked to glucosamine). For purposes of the present method any length GAG may be used.

“Purification” of oligo-GAGs may be achieved by any method commonly known in the art, including column chromatography and absorptive preparative layer chromatography.

“Lyases” as referred to herein are those members of the Enzyme Commission (EC) nomenclature group 4 which cleave glycosidic bonds (http://www.iubmb.org). Examples of lyases which have differing specificities for cleavage sites based on modifications such as sulfation and acetylation and stereoisomers such as IdoA and GlcA include Heparinase I, Heparinase II, Heparinase III, Chondroitin AC lyase, Chondroitin ABC lyase and Chondroitin B lyase.

“Separation” as used herein refers to the isolation of molecules having identical chemical composition and stereochemistry away from molecules with differing chemical composition or stereochemistry. The separation of cleavage products results in “resolution” of cleavage products.

Chromatography methods which may be used to separate and determine the modifications and stereoisomers of the cleavage products include thin layer chromatography (TLC), column chromatography, high-performance liquid chromatography (HPLC) and liquid chromatography-mass spectrometry (LC-MS).

Chromatography media which may be used in embodiments of the present invention include silica, silica gel or controlled pore glass.

A “label” as used herein refers to any moiety that enhances detection of the cleavage products. A label is optionally covalently bound to the oligo-GAG prior to treatment with lyases. Examples of labels include reporter molecules, protein conjugates, fluorescent or UV absorbing chromophores, and radioisotopes.

Where a label is applied prior to chromatography, it is preferred that the label be neutral. A charged label may be used by increasing the water component of the solvent composition by 5% for each negative charge on the label.

A “solvent” as referred to herein is a mixture which aids in the separation of cleavage products. In an embodiment of the invention, the solvent comprises an organic acid or organic derivative thereof. In a further embodiment of the invention, an organic acid or derivative thereof comprises no more than 50%, 40%, 30% or 20% by volume of the total solvent volume. In a further embodiment of the invention the organic acid is characterized by an acid dissociation constant (pKa) greater than 2.5 and smaller than 6.5. Organic acids with pKa greater than 2.5 and smaller than 6.5 include acetic acid, propanoic acid and formic acid. Examples of derivatives of organic acids with pKa greater than 2.5 and smaller than 6.5 include bromoacetic acid, iodoacetic acid, 3-chloropropanoic acid, 3-hydroxypropanoic acid and 3-butenoic acid.

The solvent optionally comprises one or more alcohols. In an embodiment of the invention, an alcohol comprises 10 or more carbon atoms and separation is performed at greater than ambient temperature, 50° F., 100° F., 150° F., 200° F., 250° F., 350° F. or 400° F. In an embodiment of the invention, an alcohol comprises no more than 9 carbon atoms and separation is performed at any temperature. Alcohol may comprise 40%, 50%, 60%, 70%, 80% by volume of the total solvent volume.

The solvent optionally comprises water. Water may comprise no more than 10%, 15%, 20% or 25% by volume of the total solvent volume. Examples of solvent compositions are listed in Table 1.

TABLE 1
Solvent Composition to Resolve Oligo-GAGs of
Different Lengths

| | Alcohol(s):Water:Organic Acid | |
|---|---|---|
| Solvent | v:v:v | Length of Oligo-GAG |
| A | 16:2:2 | 2-4 |
| B | 16:3:2 | 4-6 |
| C | 16:4:2 | 6-8 |

### EXAMPLES

### Example 1

### Preparation of GAG Derived Oligosaccharides

Oligo-GAGs were prepared by the enzymatic depolymerization of heparin according to the methods of Merchant et al. (Biochemistry Journal 229:369-376 (1985)) and Yamada et al. (The Journal of Biological Chemistry 270:8696-8705 (1995)). Heparin sodium salt from porcine intestinal mucosa (600 mg, Sigma Grade I-A; >180 USP units/mg) was dissolved in 6.0 ml of buffer A (20 mM Tris-HCl pH 7.5, 10 mM NaCl, and 1 mM CaCl₂). 50 μl of Heparinase I (IBEX 17.0 IU/ml) was added initially; two additional 50 μl portions of Heparinase I were added at 12-hour intervals. The solution was incubated at 30° C.; total incubation time was 48 hours. Aliquots of 2 and 0.2 μl were diluted into 198 and 199.8 μl of buffer A, respectively. The optical absorbencies of the aliquots were measured at 232 nm in a microtiter plate with a Spectra Max® Plus spectrophotometer (Molecular Devices, Sunnyvale, Calif.). The exhaustive digestion was achieved, and the reactions were considered complete, when the addition of enzyme failed to increase the absorbance at 232 nm.

The exhaustive digestion reaction was loaded onto a Bio-Gel® P10 gel column (1864 ml, 5×95 cm) (Bio-Rad Laboratories, Inc., Hercules, Calif.) equilibrated with buffer B: 0.2 M NH₄HCO₃ pH 8.0. The oligo-GAGs were eluted with buffer B at a 0.9 ml/min flow rate. The absorbance of the effluent was monitored at 232 nm as 7.0 ml fractions were collected. Amide 80 LC-MS was used to identify the tetra and hexa-oligosaccharide peaks. Appropriate fractions were pooled and lyophilized.

The sized oligo-GAG fractions were further purified by Source™15Q (G.E. FineLINE Pilot 35; 35/18; 173 ml) chromatography (GE Healthcare Bio-Sciences Corp., Piscataway, N.J.). The lyophilized fractions were dissolved in 25 ml buffer A (0.1M Na formate pH 3.5) and loaded onto the Source™ 15Q column equilibrated with 5 column volumes of buffer A.

The column was washed with one column volume of buffer A, followed by a linear gradient of NaCl (0 to 1.0 M) in 3500 ml of buffer A (flow rate 7 ml/min). The absorbance of the effluent was monitored at 232 nm as 7.0 ml fractions were collected.

Fractions corresponding to individual peaks were pooled and lyophilized. Each lyophilized fraction was dissolved in water and desalted on a Sephadex® G25 column (1.5×30 cm) (GE Healthcare Bio-Sciences Corp.) equilibrated with water. The product was eluted with water and the 1 ml fractions with absorbance at 232 nm were pooled and vacuum-concentrated to 0.1 to 1 μmol/ml before storage at −20° C.

### Example 2

### Labeling of Oligo-GAGs

Oligo-GAGs were labeled on the reducing end by reductive amination according to the methods described in Anumula Analytical Biochemistry, 220(2):275-283 (1994) and Bigge et al. Analytical Biochemistry, 230:229-238 (1995). Vacuum-dried oligo-GAGs (0.1 to 10.0 μM) were dissolved in 200 μl of DMSO. 20 mg of AMC, 60 μl of glacial acetic acid, and 45 mg of NaCNBH₃ was added to the carbohydrate solution. The mixture was sealed in a screw cap microfuge tube and heated to 65° C. for 24 hours followed by heating at 85° C. for 1 hour. The reaction was loaded onto a Sephadex G-25 column (1.5×30 cm) equilibrated with 0.1M NH₄CO₃. The product was eluted with 0.1M NH₄CO₃ and 1 ml fractions were collected. After assaying fractions by absorption preparative layer chromatography as described below, appropriate fractions were pooled and vacuum concentrated to a solid. The vacuum dried AMC-labeled oligo-GAG was dissolved in water and desalted on a G-25 Sephadex column (1.5×30 cm) equilibrated and eluted with water following the procedure just described.

Some oligo-GAGs were further purified by using absorption preparative layer chromatography. The vacuum dried AMC-labeled oligo-GAG was dissolved in 50% (v:v) ethanol:water and the solution was streaked onto a 1000 μm thick 20×20 cm Silica gel 60 preparative plate. Following chromatography in isopropanol:ethanol:water:glacial acetic acid (110:50:20-25:20, v:v:v:v), the appropriate band was excised and the silica crushed. The AMC-labeled oligo-GAG was eluted by washing the silica with isopropanol:water:acetic acid (4.75:4.75:0.5, v:v:v) until the silica no longer emitted fluorescence. The effluent was vacuum-dried and reconstituted in water to a concentration of 0.1 to 1 μM before storage at −20° C.

### Example 3

### Analysis of Lyase Digested Oligo-Gags by LC-MS

Vacuum-dried, purified and AMC-labeled oligo-GAGs (01. to 10.0 nmol) were reconstituted in 95 μl of solution A (20 mM Tris-HCl pH 7.5, mM NaCl, and 1 mM CaCl₂). After the addition of 5 μl Heparinase, the reaction was incubated for 18 hours at 30° C. The reaction was loaded onto a 900 μl G10 Sephadex column (poured in a 1 ml syringe with a silanized glass wool plug) equilibrated with water. The digestion products were eluted with 600 μl of water. The 100 μl flow through and 600 μl wash were combined and concentrated to a solid by vacuum filtration. All vacuum-dried samples from digests described above were reconstituted with 20 μl water followed by the addition of 180 μl acetonitrile.

The purified and AMC-labeled oligo-GAGs, and their heparinase digests, were analyzed in the negative spray mode using an Agilent 1200 series LC on-line with an Agilent 6120A Quadrapole mass spectrometer (Agilent Technologies, Santa Clara, Calif.). Using flow injection analysis, the mass spectrometer parameters were optimized for no sulfate fragmentation. The spray chamber was set with gas temperature at 295° C., nebulizer pressure at 35 psig and capillary voltage at 2000 V. The fragmentor voltage was set at 50 V. Amide-80 hydrophilic interaction liquid chromatography (HILIC) was used on the on-line LC (Naimy et al. Biochemistry 47:3155-3161(2008)) The column (TSK gel Amide 80, 20 mm ID×15 cm, 3 micron) (Tosoh Bioscience, Tokyo, Japan) was connected to a quaternary pump (G1311A) operating in gradient mode at 0.5 ml/min with solvent A (50 mM formic acid brought to pH 4.4 with ammonium hydroxide) and solvent B (Acetonitrile). After injection of 75 μl of sample, initial conditions (90% solvent B) were maintained for 1 minute. The oligosaccharides were eluted with a 33-minute linear gradient starting at 90% solvent B and descending to 40% solvent B.

### Example 4

### Digestion of Oligo-GAGs with Heparinase II and Analysis of Cleavage Products by TLC

Oligo-GAGs were analyzed by digestion with Heparinase II (IBEX, ˜17 U/ml). The digestion reactions were set up by adding 1 μl Heparinase II to a 10 μl reaction containing 0.1 to 1.0 nmol of AMC-labeled oligo-GAG in 20 mM Tris-HCl, 10 mM NaCl, and 1 mM CaCl2. Additional reactions were set up by adding 1 μl 2-O-sulfatase (Sigma-Aldrich) to 9 μl DI-S (Sigma-Aldrich) and 1 μl 2-O-sulfatase to a Heparinase II digestion reaction as described above. After incubations at 30° C. for 1 hour, 5 μl of NH₄OH (J. T. Baker, 30%) (Mallinckrodt Baker, Inc., Phillipsburg, N.J.) was added and the resealed tube was stored at 4° C. until used for TLC analyses.

A small volume of the standards and digestion reaction (2-3 μl) were spotted in a tight band on the silica gel 60 plate (without F, glass-backed) (EMD Chemicals Inc., Gibbstown, N.J.). The bands were completely dried with a hot air gun (temperature not exceeding 70° C.). For substrate concentrations less than 0.1 nmol/μl, 2-3 μl more were re-spotted and dried. The plate was developed in isopropanol:ethanol:water:glacial acetic acid (11:5:2:2, v:v:v:v), until the solvent front moved 12 to 15 cm. Following chromatography, the labeled oligo-GAGs near the origin and the digestion products that migrated further up the silica gel plate were visualized with a 314 nm UV lamp. Camera lenses were equipped with a Hoechst Blue filter (460 nm, #HB-500) (Cell Biosciences, Santa Clara, Calif.).

The developed TLC plate is shown in FIG. 5. The cleavage products in lanes 5 and 6 may be distinguished by comparison with the standard products in lanes 1 (AMC-labeled DII-S), 2 (AMC-labeled DI-S), 3 (AMC-labeled DI-S treated with 2-O-sulfatase) and 6 (labeled tetramer not treated with Heparinase II or 2-O-sulfatase). The addition of 2-O-sulfatase to DI-S-AMC (lane 3) shifted the band up to the level equal to DII-S-AMC (lane 1). This result was expected as DI-S differs from DII-S only in that it contains 2-O-sulfation; after treatment with 2-O-sulfatase DI-S should be identical to DII-S. This was confirmed as the treated DI-S and DII-S bands ran at the same level on the TLC plate.

Surprisingly, the cleavage product of labeled oligo-GAG (FIG. 3A) with Heparinase II (FIG. 5, lane 5) ran more slowly than AMC-labeled DI-S (FIG. 5, lane 2). DI-S is expected to have the same degree of sulfation as the disaccharide AMC labeled product resulting from Heparinase II digestion of the tetramer of FIG. 3A and thus was expected to travel at the same rate as DI-S. To confirm that AMC-labeled DI-S and the AMC-labeled cleavage product were identically sulfonated, the masses of the products were analyzed by mass spectrometry and found to have nearly identical masses (FIG. 4). When the cleavage product of the labeled tetrasaccharide and Heparinase II was treated with 2-O-sulfatase, the resulting band was shifted upward by a distance comparable to the shift of DI-S-AMC treated with 2-O-sulfatase (lane 3); both shifts were due to the removal of a sulfonic acid group at position 2. However, the desulfated cleavage product in lane 4, like its sulfated precursor, ran slower than the identically sulfated standard. Since the sulfation states of the standard DI-S and the desulfated cleavage product were identical (as shown by mass spectrometry and O-sulfatase treatment), the only accepted diversity was the presence of an IdoA or a GlcA.

GlcA contains the carboxyl group of C5 in an equatorial position whereas the C5 carboxyl group of IdoA is axial. The equatorial position of the C5 carboxyl group caused GlcA to move more slowly on TLC than IdoA. Lane 2 containing the faster moving, fully sulfated disaccharide contained IdoA whereas the slightly slower moving, fully sulfated disaccharide in lane 5 contains GlcA. Likewise, the faster moving, desulfated product in lanes 1 and 3 contained IdoA whereas the slower moving desulfated product in lane 4 contained GlcA.

### Example 5

### Digestion of Oligo-GAGs with Heparinase I, II & III and Analysis of Cleavage Products by TLC

Digestion reactions containing serially dilutions (1.0, 0.5 and 0.25 μl) of Heparinase I, Heparinase II and Heparinase III were prepared from aliquots of the size-selected, purified and AMC-labeled oligo-GAG tetramer illustrated in FIG. 3A. he digestion reactions were set up by adding each dilution of each Heparinase to a 10 μl reaction containing 0.1 to 1.0 nmol of AMC-labeled oligo-GAG in 20 mM Tris-HCl, 10 mM NaCl, and 1 mM CaCl2. After incubations at 30° C. for 1 hour, 5 μl of NH₄OH (J. T. Baker, 30%) (Mallinckrodt Baker, Inc., Phillipsburg, N.J.) were added and the resealed tube was stored at 4° C. until used for TLC analyses.

At completion of the digestion reaction, a small amount of the reaction mixture was spotted onto the TLC plate shown in FIG. 3B, and developed as described in Example 4.

Lanes 1 and 12 contained AMC-labeled DI-S. Lanes 2 and 13 contained AMC-labeled DII-S. DI-S and DII-S differed only in that DI-S contained 2-sulfation on the uronic acid residue; thus, the difference in the distance traveled by these two bands was a standard for movement due to a single sulfation. The remaining lanes contained AMC-labeled tetramer cleaved with serial dilutions of the following Heparinases: Lanes 4-6 Heparinase I, lanes 7-9 Heparinase II and lanes 10, 11 and 14 Heparinase III. The main cleavage products of Heparinase I and II digestion travel an equal distance on the TLC plate; however, the cleavage products of Heparinase I visibly decreased with the serial dilution of enzyme concentration whereas the cleavage products of Heparinase II appeared at approximately the same intensity regardless of the decreasing enzyme concentration. It was apparent that the difference in the activity of the enzymes was not due to the enzymes' differing specificity for sulfation or acetylation because the cleavage products of Heparinase I and II traveled at approximately the same rate on the TLC plate. The difference in travel distance made by a single sulfonic acid group is shown by the distance between the bands of lanes 1 and 2. In this case, the relevant difference in Heparinase I and II substrate specificity was the presence of GlcA in the sample. Heparinase I had a strong preference for IdoA at the cleavage site, and as the tetramer was prepared by exhaustive digestion with Heparinase I, the tetramer was not expected to contain IdoA at the cleavage site. The cleavage product detected at high Heparinase I concentration was thus the result of the very inefficient cleavage GlcA by Heparinase I; the amount of labeled cleavage product detected decreased substantially with reduced concentration of the enzyme. In contrast Heparinase II was not affected by IdoA or GlcA at the cleavage site and thus as the enzyme concentration was decreased, the availability of Heparinase II to contact a suitable tetramer substrate was not diminished nor was the presence of the AMC-labeled cleavage product.

### Example 6

### Digestion of a Mixture of Oligo-Gags with Heparinase I, II & III and Analysis of Cleavage Products by TLC

Digestion reactions containing serially dilutions (1.0, 0.5 and 0.25 μl) of Heparinase I, Heparinase II and Heparinase III, respectively, were prepared from aliquots of the size-selected, purified and AMC-labeled oligo-GAG tetramer mixture illustrated in FIG. 6A. The digestion reactions proceeded as described in Example 5, except that the AMC-labeled oligo-GAG mixture of FIG. 6A was used instead of the AMC-labeled oligo-GAG of FIG. 3A. At completion of the digestion reaction, a small amount of the reaction mixture was spotted onto the TLC plate shown in FIG. 6B and developed as described in Example 4. Lanes 1 and 13 contained AMC-labeled DI-S. Lanes 2 and 13 contained AMC-labeled DII-S. DI-S and DII-S differed only in that DI-S contained 2-sulfation on the uronic acid residue, thus the difference in the distance traveled by these two bands was a standard for movement due to a single sulfation. The remaining lanes contained AMC-labeled tetramer cleaved with serial dilutions of the following Heparinases: lanes 3-5 Heparinase I (17.0 IU/ml), lanes 7-9 Heparinase II (15.2 IU/ml) and lanes 10-12 Heparinase III (17.2 IU/ml). The absence of any Heparinase I cleavage products, when both Heparinase II and III both produced cleavage products, indicated that the tetramers did not contain a 2-O-sulfate group on the uronic acid component. Heparinase II digestion resulted in two, closely spaced bands. The faster band co-migrated the same distance as the AMC-labeled DII-S (lane 2) and the slower band co-migrated the same distance as the product in lane 4, FIG. 5 (β-D-GlcA-(1-4)-D-GlcNS6S-AMC). Heparinase II cleaved when either form of uronic acid was present at the cut site, whereas Heparinase III only cleaved GlcA in this context. The Heparinase III digestion produced only the slower migrating band of the two closely spaced bands produced by the Heparinase II digest. The difference in specificity of Heparinase II and the Heparinase III enabled the identification of the two closely spaced bands. The upper of the two bands produced only by Heparinase II contained IdoA at the cut site; the lower band produced by Heparinase II and III contained GlcA at the cut site.

Digestion reactions containing serially dilutions (1.0, 0.5 and 0.25 μl) of Heparinase I, Heparinase II and Heparinase III, respectively, were prepared from aliquots of the size-selected, purified and AMC-labeled oligo-GAG tetramer mixture illustrated in FIG. 7A. Both GAG tetramers had 3 sulfates and one acetyl group as calculated by mass. The digestion reactions proceeded as described in Example 5, except that the AMC-labeled oligo-GAG mixture of FIG. 7A was used instead of the AMC-labeled oligo-GAG of FIG. 3. At completion of the digestion reaction, a small amount of the reaction mixture was spotted onto the TLC plate shown in FIG. 7B and developed as described in Example 4. Lane 1 contained the tetramer mixture illustrated in FIG. 7B. Lane 11 contained AMC-labeled DI-S. Lane 12 contained AMC-labeled DII-S. The remaining lanes contained the AMC-labeled tetramer mixture cleaved with serial dilutions of the following Heparinases: lanes 2-4 Heparinase I (17.0 IU/ml), lanes 5-7 Heparinase II (15.2 IU/ml) and lanes 8-10 Heparinase III (17.2 IU/ml). No Heparinase I or II cleavage products (lanes 2-7) were observed since the glucosamine residue at the cut site was acetylated and there was no sulfation at the amine. The two bands that were observed in lanes 2-7 were the resolution of the uncut tetramers based on their containing IdoA at the cut site or GlcA at the cut site. With the absence of sulfation at the adjacent glycosamine, Heparinase III had activity for sites containing iduronic acid (Wei Z., et al. The Journal of Biological Chemistry 280:15742-15748 (2005)). Heparinase III digestion showed two fragments due to the variable sulfation at the non-reducing glucosamine.

1. A method for determining a stereochemistry for a component of a composition, the composition further comprising a glycosaminoglycan (GAG), wherein the method comprises:
a. cleaving the GAG with a lyase;
b. separating the products of the cleaved GAG by chromatography in the presence of one or more solvents wherein at least one solvent comprises an organic acid or organic acid derivative in an amount of no more than 50% v:v of solvent; and
c. determining the stereochemistry of the component.

2. A method according to claim 1, wherein the lyase is selected from the group consisting of Heparinase I, Heparinase II, Heparinase III, Chondroitin ABC lyase, Chondroitin AC lyase and Chondroitin B lyase.

3. A method according to claim 1, wherein the GAG is modified and the modification comprises sulfation, carboxylation or acetylation.

4. A method according to claim 1, wherein a component comprises a uronic acid or hexosamine or both.

5. A method according to claim 1, wherein the composition is isolated from a tissue of an organism.

6. A method according to claim 1, wherein the composition is synthetic.

7. A method according to claim 1, wherein the composition is a polysaccharide, glycoprotein or glycolipid.

8. A method according to claim 1, wherein chromatography includes column chromatography, thin layer chromatography and high-performance liquid chromatography.

9. A method according to claim 1, further comprising subjecting the composition to mass spectrometry.

10. A method according to claim 1, wherein the organic acid or organic acid derivative has an acid disassociation constant (pKa) of less than 2.5 and no more than 6.5.

11. A method according to claim 10, wherein the organic acid is selected from the group consisting of acetic acid, formic acid and propanoic acid.

12. A method according to claim 1, wherein the at least one solvent comprises one or more alcohols.

13. A method according to claim 1, wherein the at least one solvent additionally comprises at least 50% alcohol (v:v).

14. A method according to claim 1, wherein the chromatography media is silica, silica gel or controlled pore glass.

15. A method according to claim 1, wherein the one or more solvents further comprise an acetonitrile solvent.
